(19) United States
(12) Patent Application Publication
(10) Pub. No.: US 2015/0192557 A1
(43) Pub. Date: Jul. 9, 2015

(54) TELEMETRY SYSTEM FOR DISTRIBUTED SOIL MOISTURE CONTENT MEASUREMENT

(71) Applicant: Ananke Enterprises Pty Ltd, Box Hill (AU)

(72) Inventor: Nemo Bilus Abaffy, Box Hill (AU)

(21) Appl. No.: 14/590,230

(22) Filed: Jan. 6, 2015

(30) Foreign Application Priority Data
Jan. 6, 2014 (AU) 2014900023

(51) Int. Cl. G01N 33/24; G06T 19/00

(52) U.S. Cl. CPC G01N 33/246; G06T 19/006; G06T 2215/16; G06T 2207/30188

(57) ABSTRACT

A telemetry system for distributed soil moisture content measurement, the system comprising: a data network adapted for transmitting data; a sensor operably coupled to the data network; a computing device operably coupled to the data network adapted for providing an augmented reality display; and an interface computing device adapted to communicate with the computing device, wherein, in use: the sensor is adapted to generate sensor data representing a measurement; the interface computing device is adapted to receive, from the sensor, the measurement data and communicate the measurement data to the computing device; and the computing device is adapted to process the measurement data received from the interface computing device; convert the measurement data into augmented reality data and to display the augmented reality data on a display module of the computing device representing an augmented reality display in accordance with the sensor data.

### CROSS-REFERENCE TO RELATED APPLICATIONS

This application claims the benefit of Australian Provisional Patent Application Serial No. AU2014900023 entitled “A TELEMETRY SYSTEM FOR DISTRIBUTED SOIL MOISTURE CONTENT MEASUREMENT” filed on Jan. 6, 2014. The entirety of the above-noted application is herein incorporated by reference.

1. Field of the Invention

The present invention relates to telemetry systems and in particular to a telemetry system for distributed soil moisture content measurement.

The invention has been developed primarily for use in soil moisture content measurement and will be described hereinafter with reference to this application. However, it will be appreciated that the invention is not limited to this particular field of use. Specifically, the invention may be used for measuring other measurements also, including those specifically related to the agricultural industry.

Furthermore, the invention preferably allows for the augmented reality display of the soil moisture content measurement. However, it should also be appreciated that the invention is not limited to necessarily having to display in augmented reality.

2. Background

Any discussion of the background art throughout the specification should in no way be considered as an admission that such background art is prior art nor that such background art is widely known or forms part of the common general knowledge in the field in Australia or worldwide.

According to existing arrangements, irrigation systems are used in agriculture, such as for use in vineyards and the like for attempting to ensure that soil is kept at optimum moisture content for growing purposes.

Such irrigation systems generally run according to a schedule, such as, for example, wherein the irrigation system is operated for an hour each morning.

However, such scheduling of irrigation fails to take into account environmental considerations such as evaporation. In this manner, situations can arise where the soil is not moist enough or alternatively wherein the soil is over irrigated thereby potentially destroying the vines and wasting water.

In this manner, sensors are employed for the purposes of controlling such irrigation. However, these existing arrangements often times treat the entire irrigation area uniformly wherein the decision to irrigate or not is usually obtained from one or more sensor adapted to obtain indicative soil moisture content measurements. As such, these existing arrangements fail to take into account variations across the entire irrigation area wherein, for example, certain areas may be over irrigated with other areas becoming dry.

Furthermore, existing irrigation systems fail to provide soil moisture content measurements in a comprehendible manner, if at all. As such, operators, given the paucity of data, are ill equipped to ascertain the irrigation state of an irrigation area.

It is to be understood that, if any prior art information is referred to herein, such reference does not constitute an admission that the information forms part of the common general knowledge in the art, in Australia or any other country.

### SUMMARY OF THE INVENTION

It is an object of the present invention to overcome or ameliorate at least one or more of the disadvantages of the prior art, or to provide a useful alternative.

According to one aspect, there is provided a telemetry system for distributed soil moisture content measurement. The system may comprise a data network adapted for transmitting data. The system may further comprise one or a plurality of sensors operably coupled to the data network. The system may further comprise a computing device operably coupled to the data network adapted for providing an augmented reality display. The system may further comprise an interface computing device may be adapted to communicate with the computing device. In use, the sensors may be adapted to generate sensor data representing a measurement. In use, the interface computing device may be adapted to receive, from the sensors, the measurement data. The interface computing device may be adapted to communicate the measurement data to the computing device. The computing device maybe adapted to process the measurement data received from the interface computing device. The computing device may be further adapted to convert the measurement data into augmented reality data. The computing device may be further adapted to display the augmented reality data on a display module of the computing device representing an augmented reality display in accordance with the sensor data.

According to an arrangement of the above aspect, there is provided a telemetry system for distributed soil moisture content measurement, the system comprising a data network adapted for transmitting data; as one or a plurality of sensors operably coupled to the data network; a computing device operably coupled to the data network adapted for providing an augmented reality display; and an interface computing device adapted to communicate with the computing device, wherein, in use: the sensors are adapted to generate sensor data representing a measurement; the receiver device is adapted to receive, from the sensors, the measurement data and communicate the measurement data to the computing device; and the computing device is adapted to process the measurement data received from the interface computing device; convert the measurement data into augmented reality data and to display the augmented reality data on a display module of the computing device representing an augmented reality display in accordance with the sensor data.

The sensors may be adapted for sensing moisture content.

The augmented reality display may represent the measurement.

The augmented reality display may represent a binary threshold representation of the measurement.

The augmented reality display may represent a scalar representation of the measurement.

In use, the augmented reality display device may be adapted to display the sensor data in accordance with a location of the sensors.

The augmented reality display device may be further adapted for receiving, from the sensors, location data representing a location of the sensors.

In use, the augmented reality display device may be configured with a location of the sensors.

The augmented reality display device may be adapted to display an interpolation of the measurement.

The interface computing device may be operably coupled to the data network. In use, the augmented reality display computing device may be adapted to receive, from the sensors via the interface computing device, the measurement data.

The system may further comprise drop in network routers and/or repeaters to be able to extend the range of the sensor network to the interface computing device. The routers and/or repeaters may comprise battery management circuitry. The routers and/or repeaters may comprise an electrical power generation module. The electrical power generation module may comprise one or more solar panels such that the routers and/or repeaters may remain in an active, powered on, state for extended periods of time. Use of such routers and or repeaters and the like additionally provides the significant advantage that the telemetry system disclosed herein is not necessarily limited by available connectivity to existing cellular network communications systems and thus is not reliant on available mobile phone network coverage.

The interface computing device may comprise memory for storing digital data. In use, the interface computing device may be adapted to cache the sensor data.

In use, the interface computing device may be adapted to establish communication with the augmented reality display computing device when the augmented reality display computing device is within proximity of the interface computing device and, having establish communication, transmit, to the augmented reality display computing device, sensor data from the memory.

Preferably, the interface computing device is adapted to interface with the sensors using a first wireless protocol and interface with the augmented reality display computing device using a second wireless protocol.

The first and or the second wireless communication protocol may comprise either Bluetooth Low Energy (BLE) or internet protocol (e.g. IPv4 or IPv6)to enable direct communications between the interface computing device and with the computing device and/or the sensors directly to better ascertain relative position of the sensors. The relative position of the sensors may be calculated using received signal strength indication (RSSI) and/or time of flight of telemetry data between the interface computing device and the sensors.

In particular arrangements, the first wireless protocol may be the ZigBee wireless protocol.

In particular arrangements, the second wireless protocol may be the 802.11 wireless protocol.

Preferably, the interface computing device comprises a web server and wherein, in use the interface computing device is adapted to receive an HTTP request from the augmented reality display computing device; and the interface computing device is adapted to send, to the augmented reality display computing device, an HTTP response comprising the sensor data.

Preferably, in use, the interface computing device is adapted to send, to a remote data server, the sensor data.

Preferably, in use, the augmented reality display computing device is adapted to receive, from the data server, the sensor data.

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a computing device on which the various embodiments described herein may be implemented in accordance with an embodiment of the present invention; and

FIG. 2 shows telemetry system 200 for distributed soil moisture content measurement on which the various embodiments described herein may be implemented in accordance with an embodiment of the present invention.

FIG. 3 is an example icon representation of a moisture sensor applicable to the telemetry systems disclosed herein in accordance with an embodiment of the present invention.

### DETAILED DESCRIPTION OF EMBODIMENTS

In the description below, there will be described telemetry system for distributed soil moisture content measurement. In particular, in accordance with an exemplary embodiment, there will be described the use of the telemetry system by a vigneron and the monitoring of the soil moisture content of a vineyard.

Furthermore, in an especially preferred embodiment, the telemetry system is adapted for the display, in augmented reality, of the real-time soil moisture content measurement. In this manner, the vigneron may walk about the vineyard so as to view, in substantial real time, the moisture content state of the vineyard in an intuitive and user-friendly manner.

Of course, it should be appreciated that the technical embodiments described herein may be suited for other applications other than soil moisture content measurement. Specifically, the system may measure other measurements as opposed to soil moisture content such as, within the agricultural industry, rainfall, sunlight, wind-speed, temperature, electrical conductivity of the soil and the like.

Furthermore, the embodiments described herein need not necessarily be used specifically within the viticulture industry and may be used in industries also, including other agricultural industries such as cereal crop cultivation and the like. Indeed, the system disclosed herein need not necessarily be utilised within the agricultural industry at all, and may, for example, be used in other industries such as wherein a civil engineer is able to view, in augmented reality, the stress state of various girders of a bridge in use.

Furthermore, it should be noted that the telemetry system need not necessarily support augmented reality in all embodiments in that embodiments of the telemetry system described herein are commercially practicable without necessarily requiring augmented reality.

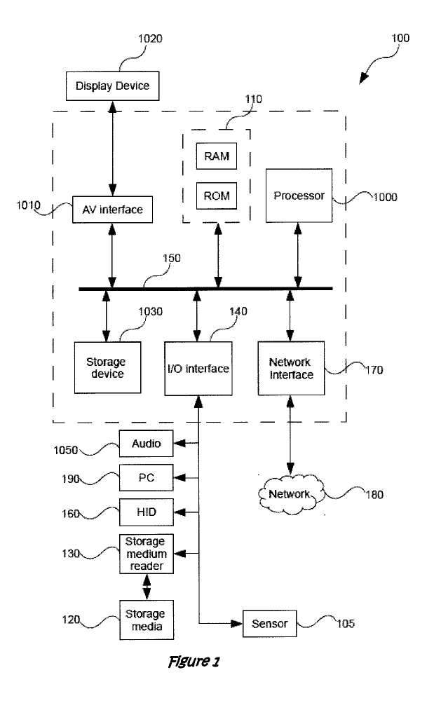

Turning now to FIG. 1, there is shown a computing device 100 on which the various embodiments described herein may be implemented.

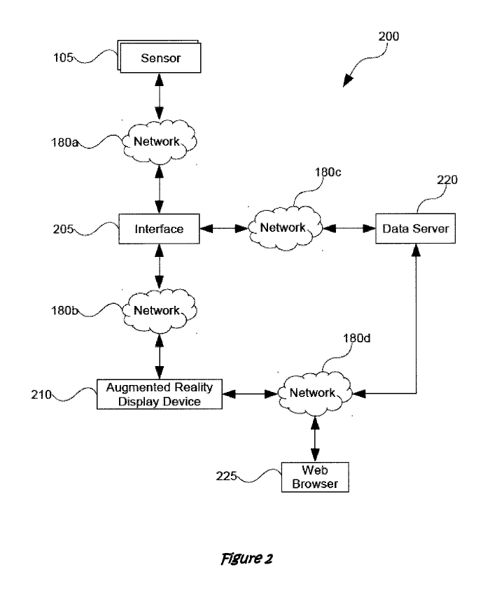

The computing device 100 may take on the different computing device embodiments, especially those described in FIG. 2. Specifically, and as will be described in further detail below, the telemetry system may, in different embodiments, comprise a plurality of sensors, an augmented reality display device for displaying in augmented reality the moisture soil content, an interface computing device between the sensors on the augmented reality display device, a data server and a client computing device comprising a browser application.

It should be noted that the typical integers of the computing device 100 are exemplary only and that each technical integer of the telemetry system described in further detail below need not comprise each and every technical integer enumerated in FIG. 1. For example, where the sensor comprises the computing device, the sensor need not necessarily comprise a display device.

The steps of the method of measuring soil moisture content, generating augmented reality display data and the like may be implemented as computer program code instructions executable by the computing device 100. The computer program code instructions may be divided into one or more computer program code instruction libraries, such as dynamic link libraries (DLL), C++ static lib arrays and header files, Java Archive (JAR) and the like, wherein each of the libraries performs a one or more steps of the method. Additionally, a subset of the one or more of the libraries may perform graphical user interface tasks relating to the steps of the method. In particular arrangements, the generation of the visualisation (e.g. augmented reality display) display data may be executed by code instructions executable by either centralized data server 220 (as seen in FIG. 2) or, if hardware computing components of the augmented reality computing device are sufficient, the visualization display data may be executed by code instructions executable by the augmented reality computing device 210 itself. Generation of the visualisation display data by the augmented reality computing device 210 itself may prove advantageous by minimizing the load on the centralized server 220 in the situation where the server 220 is adapted to process requests from a plurality of augmented reality computing devices 210. Rendering the visualisation data on the device is also advantageous in remote areas where mobile communications connectivity is scarce, thus allowing the augmented reality visualisation to be rendered without direct access to the centralized data server 220.

In further arrangements, the augmented reality view of the sensors of the telemetry system may be offered as a standalone library that can be incorporated with other platforms, which may then be released under a dual licensing scheme; GPL and commercial license or similar arrangement.

The visualization data generation may be executed on the server 220 using, for example, javascript program rendering or the like. The visualization data generation may be executed on the augmented reality computing devices 210 using, for example, openGL program rendering or the like. The device 100 comprises semiconductor memory 110 comprising volatile memory such as random access memory (RAM) or read only memory (ROM). The memory 100 may comprise either RAM or ROM or a combination of RAM and ROM.

The device 100 comprises a computer program code storage medium reader 130 for reading the computer program code instructions from computer program code storage media 120. The storage media 120 may be optical media such as CD-ROM disks, magnetic media such as floppy disks and tape cassettes or flash media such as USB memory sticks.

The device further comprises I/O interface 140 for communicating with one or more peripheral devices. The I/O interface 140 may offer both serial and parallel interface connectivity. For example, the I/O interface 140 may comprise a Small Computer System Interface (SCSI), Universal Serial Bus (USB) or similar I/O interface for interfacing with the storage medium reader 130. The I/O interface 140 may also communicate with one or more human input devices (HID) 160 such as keyboards, pointing devices, joysticks and the like. The I/O interface 140 may also comprise a computer to computer interface, such as a Recommended Standard 232 (RS-232) interface, for interfacing the device 100 with one or more personal computer (PC) devices 190. The I/O interface 140 may also comprise an audio interface for communicate audio signals to one or more audio devices 1050, such as a speaker or a buzzer.

The device 100 also comprises a network interface 170 for communicating with one or more computer networks 180. The network 180 may be a wired network, such as a wired Ethernet™ network or a wireless network, such as a Bluetooth™ network or IEEE 802.11 network. The network 180 may be a local area network (LAN), such as a home or office computer network, or a wide area network (WAN), such as the Internet or private WAN.

The device 100 comprises an arithmetic logic unit or processor 1000 for performing the computer program code instructions. The processor 1000 may be a reduced instruction set computer (RISC) or complex instruction set computer (CISC) processor or the like. The device 100 further comprises a storage device 1030, such as a magnetic disk hard drive or a solid state disk drive.

Computer program code instructions may be loaded into the storage device 1030 from the storage media 120 using the storage medium reader 130 or from the network 180 using network interface 170. During the bootstrap phase, an operating system and one or more software applications are loaded from the storage device 1030 into the memory 110. During the fetch-decode-execute cycle, the processor 1000 fetches computer program code instructions from memory 110, decodes the instructions into machine code, executes the instructions and stores one or more intermediate results in memory 100.

In this manner, the instructions stored in the memory 110, when retrieved and executed by the processor 1000, may configure the computing device 100 as a special-purpose machine that may perform the functions described herein.

The device 100 also comprises a video interface 1010 for conveying video signals to a display device 1020, such as a liquid crystal display (LCD), cathode-ray tube (CRT) or similar display device.

The device 100 also comprises a communication bus subsystem 150 for interconnecting the various devices described above. The bus subsystem 150 may offer parallel connectivity such as Industry Standard Architecture (ISA), conventional Peripheral Component Interconnect (PCI) and the like or serial connectivity such as PCI Express (PCIe), Serial Advanced Technology Attachment (Serial ATA) and the like.

Turning now to FIG. 2, and as alluded to above, there is disclosed a telemetry system 200 for distributed soil moisture content measurement. As also alluded to above, while in arrangements the telemetry system 200 is adapted for soil moisture content measurement, and in arrangements in the augmented reality display of soil moisture content, it should be noted that the telemetry system 200 may be applicable for use in other applications also, including those not specifically relating to agriculture.

The system 200 comprises a plurality of sensors 105, which, in the preferred embodiment described herein are adapted for measuring soil moisture content, however the sensors may be adapted for alternative application(s) in accordance with requirements. Generally, the plurality of sensors 105 would be distributed in grid formation about a vineyard area so as to provide coverage for the entire vineyard. The spacing of the sensors may be adjusted in accordance with the desired resolution of soil content measurement. The sensors may be optimised for low cost sensor design and battery life. For example, sensor cost and operating requirements can be minimised using, for example, capacitive sensors with quick response time and low operating voltages allow for a minimalist sensor circuit design in contrast to existing sensor products available which rely on complex circuitry that makes their costs prohibitive for many small applications of the presently disclosed telemetry system.

During the installation of the sensor devices which are expected to be static (i.e. operate in a fixed location), the installer is able to specify a descriptive name which will be associated with the sensor, minimum/maximum values expected for the sensor and most importantly, the installer is able to record the physical location of each sensor according to the GPS receivers as typically available on mobile computing devices adaptable to interface with the presently disclosed telemetry system. This provides significant operational advantages to the presently disclosed systems which increases the usability of the system as well as obviating the need for the sensor to contain power hungry and costly GPS receiver or other location enabling circuitry components.

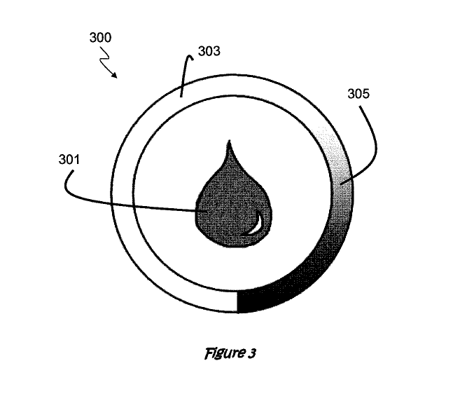

In practical use, the system is designed with user/installer/operator ease of use in mind. For instance, during the initial setup phase of the telemetry system, the system may be designed such that, for example, when the interface computing device 205 is connected to the system e.g. via USB or alternative suitable connection protocol, the user is directed to install the associated software application on the user's preferred computing device 210 such as a mobile computing device such as e.g. a tablet or smartphone device. At this point the sensors are automatically registered and their sensor information is being logged by the server 220 and/or the computing device 210. If the computing device 210 has a network connection, then the device data is automatically sent to the server 220. Any device 210 using the augmented visualisation library is now able to visualise the sensor data. The visualisation represents the type of sensor (specific icon) and the soil moisture level (or sensor specific data) in a readily apparent way. For instance, particular design aspect of the data visualisation components of the system are particularly suited to enablement of examiner mentions are critical to the commercial viability of the system. They enable low cost, low power sensors leveraging ubiquitous smartphone devices consumers already have and are familiar with. Readily apparent to the user is the physical location of the sensor in relation to them with the augmented reality view in a way similar to some video games' minimap. The type of sensor is also made apparent to the user by efficient use of iconography. The icon may also be adapted to include a representation of the measurement data provided by the actual sensor being represented and the sensor data representation can take into account minimum and maximum values determined either by the user or default to the individual sensor. For example, as depicted in FIG. 3, there is provided an example icon representation of a moisture sensor applicable to the telemetry systems disclosed herein. The icon 300 comprises a sensor identifier 301 which in the present example includes an image of a water droplet to depict that the sensor provides sensor data related to moisture or water content in a sample or location associated with the sensor. In this particular example, the sensor data is depicted in a ring area 303 around sensor identifier 301. Sensor data area 303 may be colour coded and/or filled in such a way as to most readily indicate the sensor information e.g. as a gradient representation 305. The state of the sensor and its sensor data depicting the sensor environment is readily apparent to the user from the colour code which in particular arrangements, may be adapted for users with normal colour vision and for the colour blind alike, and the filling of the ring which may be indicative of the minimum and maximum values expected for the particular sensor.

In further arrangements, the sensor iconographic representation may be further adapted to include a depiction of historical sensor data such as the range/variability of values within the last week/month/year depending on user preferences.

In preferred arrangements, the user is able to view a much more in-depth view of the sensor information or sensor data either by touching it on the screen or minimap or centering on it in the visualisation display on the computing device 210 (details of sensor in centre may advantageously be displayed in pop-up view). In the case of a computing device 210 comprising a Google Glass™ device, the user may be able to navigate and select the sensors on the minimap by holding on to an associated input device such as a touchpad and moving their head to alter the augmented reality view.

Upon selecting or entering into the sensor, the sensor's historical data may advantageously be presented to the user. The user may then optionally be able to manipulate the historical time series chart by swiping to see values at other times, using two finger pinching in to zoom in and out to zoom out to the appropriate values vs time.

From this screen the user is able to enter a settings screen to enable modification of the display data properties to the use's preferences, for example, the user may able to set location, min, max, name etc. for particular sensor representations or to include alternative views, potentially as additional data overlays to the augmented reality display. Such alternative views of the sensor may either be overlayed over the sensor icon or take up the screen depending on the available processing power of the user's computing device 210

As would be appreciated, effective presentation of the sensor information is crucial to the augmented reality aspect of the telemetry systems disclosed herein. It invites the user's curiosity or provides them with the minimum level of data required in the most efficient and time-sensitive manner without burdening them with too much information. Effective visualisation of the sensor information allows the user to visually analyse the information and quickly distill it for application in subsequent decision making activities.

In a particular example, the sensors 105 may be located adjacent to vineyard posts or other support means. Alternatively, the sensors 105 may be located anywhere within the vineyard and potentially submerged so as to avoid damage. In this embodiment, the sensors 105 may comprise a flexible antenna or the like so as to be to wirelessly transmit measurement data from the sensor 105 beneath the soil without radiofrequency attenuation.

Preferably, the sensor 105 is a low-cost sensor having a ruggedised construction suited for field use. Preferably, the sensor 105 measures the dielectric constant of the soil using transmission line techniques so as to be insensitive to water salinity. Further preferably, the sensor 105 does not utilise conductivity based probe measurement technique so as to substantially overcome corrosion.

In a preferred embodiment, the sensor 105 comprises a radio transceiver adapted to communicate wirelessly using a radio transmission protocol across a wireless network 180, such as short-range Bluetooth, Zigbee or 802.11 protocols, or longer range media transmission protocol such as cellular based wireless communication including 3G.

In a further arrangement, the system may further comprise one or a plurality of further network architecture modules such as, for example, wireless routers, signal boosters, repeaters and the like, adapted to extend the range of the wireless communications networks. The routers and/or repeaters may comprise an electrical power generation module. The network architecture modules may comprise one or more solar panels such that the network architecture modules may remain in an active, powered on, state for extended periods of time.

Alternatively, in further embodiment, the sensor 105 may be a wired sensor 105 as opposed to a wireless sensor 105. In this manner, the sensor 105 may comprise a suitably long measurement lead or the like allowing for the coupling of the sensor 105 to an appropriate computing device, such as the augmented reality display device 210 or the interface computing device 205 as will be described in further detail below.

In a particular example arrangement disclosed herein, the sensors 105 may be configured to measure soil or water content in different manners. In one manner, the sensors 105 may be adapted to perform the measurement of soil moisture content at periodic intervals, such as upon the hour. It is, however, to be appreciated that the system may be readily adaptable to many other application examples in accordance with requirements. Furthermore, the sensors 105 may comprise a memory device 110 or the like so as to store the measurement of soil moisture content.

In particular example arrangements, the presently disclosed systems is adapted to utilize and exploit the ubiquity of commoditised components, such as existing sensor devices, and be compatible with many different types of mobile computing devices, e.g. smartphones, tablets and the like.

In this manner, each sensor 105 may comprise a historical soil moisture content measurement comprising a plurality of soil moisture content measurements spaced apart in time. In this manner, the sensor 105 advantageously allows not only for the present soil moisture content measurement representation but also the historical soil moisture content measurement so as to allow a vigneron to view the rate of evaporation, wetting or the like. Generally, where historical moisture content measurement recorded by each sensor 105, each sensor 105 may comprise sufficient memory to record water moisture content data for an appropriate time period, such as a week or a month, depending on the application.

Furthermore, each sensor 105 may be adapted to periodically transmit the soil moisture content measurement data. In this manner, upon receipt of each moisture content measurement, or after a certain amount of moisture content measurements have been made by the sensor 105, the sensor 105 may transmit the measurement of measurements across the network 180 to the other computing devices 100 within the system 200.

In an alternative embodiment, as opposed to the sensors 105 automating the recording of soil moisture content measurements in an automated manner, in one embodiment, the sensors 105 may be adapted to record soil moisture content measurements upon demand. As will be discussed in further detail below, in one embodiment, the telemetry system 200 comprises an interface computing device 205 adapted for wireless or wired communication with the plurality of sensors 105. In this manner, upon request from the interface computing device 205, or even when the interface computing device 205 comes within range of the sensors 105, the sensors 105 may then be adapted to record soil moisture content measurements.

In a yet further embodiment as also be described in further detail below, the telemetry system 200 comprises a web browser 225 such as a client computing device operably coupled to the Internet. In this manner, using the web browser 225, the vigneron may request the recording of the soil moisture content measurements wherein an instruction is sent to each remote sensor 105 to initiate a recording of such moisture content measurements for return to the web browser 225. In this manner, the vigneron may receive substantially real time snapshot of the moisture content status of his vineyard.

Now, in the above-mentioned preferred embodiment, the telemetry system 200 is adapted for the display of the soil moisture content, and further preferably in an augmented reality manner, such as by using an augmented reality display device 210.

However, the first embodiment will now be discussed wherein the system 200 is simply adapted for displaying the soil moisture content measurements in a two-dimensional manner. In this manner, the display device 210 (not having augmented reality capabilities in this embodiment) is adapted to display a two-dimensional map of the vigneron's vineyard wherein the representation of the moisture content measurements is represented in accordance with the location of each sensor.

In this manner, the vigneron may be able to view which areas of the vineyard are not at optimal moisture content requiring either less or more water application.

In one embodiment the system 200 may be operably coupled to an irrigation system (not shown) and especially an irrigation system having discreetly controllable irrigation outputs so as to be able to irrigate specific portals of the vineyard. In this manner, upon detection of a deficit of moisture content in a particular region of the vineyard, the irrigation system may be controlled by the system 200 to irrigate the relevant region of the vineyard.

In representing the soil moisture content, the system 200 may display the soil moisture content in a number of manners. In one manner, the display may simply represent a binary thresholds representation of the water content measurement. For example, areas that have greater than the optimal water content may be represented in a transparent blue overlay overlaid the map representation whereas areas having less than optimal water content have no overlay.

However, in a preferred embodiment the moisture content measurements may be represented at a scalar manner so as to represent a soil moisture content scale from dry, being represented with no overlay, to saturated, being represented by a dark blue overlay.

Given that the sensors 105 are spaced apart about the vineyard, the system 200 may be configured to employ interpolation technique to estimate moisture content measurements between sensors 105. In this manner, the display of the moisture content measurements may be smooth and represented without an awareness by the vigneron as to the actual locations of each sensor 105.

In a further embodiment, the system 200 may be adapted to estimate future soil moisture content measurements in accordance with differing environmental factors affecting the rate of evaporation. For example, on especially warm or sunny days the system 200 would apply a higher evaporation rate as opposed to were it colder or less windy. In this manner, the vigneron may view the current soil moisture content measurements and estimation as to future soil content measurements. In one embodiment, the system 200 may comprise a weather station or the like (not shown) adapted for the purpose of obtaining these measurements.

However, as alluded to above, in a preferred embodiment, the system 200 is adapted for the augmented reality display of the soil moisture content measurements from each sensor 105. As such, the system 200 comprises the augmented reality display device 210 which is adapted to display the soil moisture content measurements from its sensor 105 in substantial augmented reality.

In this manner, the vigneron they simply walk about his vineyard, and, utilising the augmented reality display device 210, be able to view which areas of his vineyard require watering in substantial real time.

The augmented reality display device 210 may take the form of a low-cost consumer computing device, such as a tablet computing device such as, for example, an Apple iPad or the like or more advanced augmented reality display device 210 such as augmented reality headsets such as the Google Glass™ augmented reality headset or the like. In this manner, the overall cost of the system 200 may be kept low.

In displaying the augmented reality display of the soil moisture content measurements from each sensor 105, the augmented reality display device 210 must first determine the location of the augmented reality display device 210 and the orientation of the augmented reality display device 210 so as to be able to overlay the soil moisture content measurement data over image or video data captured by the augmented reality display device 210.

The position or location of each of the sensors 105 using a GPS receiver associated with each sensor 105 wherein the sensor communicates its physical location to the interface computing device 205 over the network 180a. Interface computing device then communicates the GPS location data received from the sensor 105 to the computing device 210 which, in turn, calculates the sensor location for using the augmented reality (or otherwise) display.

In alternative arrangements, the physical location of each sensor may be recorded directly in the computing device at the initial setup and configuration stage of the system. For example, the computing device 210 may comprise a GPS receiver which a user may use to record the location of each sensor as it is placed into position. This may be particularly advantageous to enable minimization of the fabrication and operating costs for each sensor i.e. by obviating the need for a separate GPS receiver in each individual sensor. Alternatively still, the system may utilize further position locating methods for determining the physical location of each sensor 105, for example returned signal strength indication (RSSI) and time of flight are alternative ways to determine proximity and relative location when used in conjunction with other sensor devices using either ZigBee or Bluetooth LE communication protocols. Further still, the system may utilize Kalman filtered combinations of inertial navigation system hardware available on the computing device (including one or more of magnetometers, gyroscopes, accelerometers or alternatives and other inbuilt internal inertial navigations components available in the computing device as would be appreciated by the skilled addressee). The signals from each of the internally available components may be assigned different weighting factors in accordance with the usefulness in improving the user experience in viewing the visualization data on the display screen of the computing device and/or determining the position of the telemetry sensor(s) 105 relative to computing device 210:

The collection of typically available internal location/position sensors of the computing device may also be utilized to improve the user experience when viewing the generated visualization data. For instance, internal sensors of the computing device may be used for such purposes as providing less jerky movements of the sensor on the display screen of the computing device, minimising apparent sensor drift, etc.

The data server 220 may hold both private and publicly available sensor data which may be searched by a user either by way of location or sensor type or another search parameter. In the case of a private network, authentication is made either by way of a preconfigured system 200 and/or by a standard authentication service/method.

In particular, the addition of Bluetooth Low Energy (BLE) componentry to smartphone and tablet computing devices on the market has enabled more accurate positioning of the sensors themselves at smaller distances to the BLE enabled computing device. This is a great complement to the relative location as determined by GPS/network, as gps/network triangulation suffers at close proximity and/or inside buildings. By obtaining the RSSI and/or time of flight of data received from multiple sensors and combining (differently weighting) them with GPS/network obtained relative location (latitude longitude difference taking into account earth curvature etc.) according to proximity between device and sensor a much more accurate overlay of the sensor representation is able to be rendered. In the case of a static sensor, a further improvement in the accuracy of the sensor representation overlay is able to be achieved by calibrating RSSI/time of flight to take into account the attenuation of the signal strength and/or change in time of flight and/or specific network path due to the physical environment of the sensor in relation to the computing device.

Generally, for soil moisture content measurements the system 200 will ascertain a ground plane from the above location and orientation information representing the ground surface of the vineyard and overlay upon the ground plane the soil moisture content measurements.

In an alternative embodiment, the system 200 may augment the display of the actual vines themselves wherein, for example, a vine requiring water will be coloured red whereas a vine having sufficient water will be represented in green or blue.

There are a number of manners in which the augmented rally to display device 210 may ascertain the location of the augmented reality display device 210. In one manner, the augmented reality display device 210 comprises a location sensing means such as an inbuilt GPS sensor so as to be able to determine the location of the augmented reality display device 210. Furthermore, the augmented reality display device 210 may comprise a solid state tri-axial gyroscope or the like to ascertain the orientation of the augmented reality display device 210.

An alternative embodiment, the augmented reality display device 210 may utilise image reference markers to determine the location and orientation of the augmented reality display device 210. For example, each vineyard post may have a coloured ball such as a ping-pong ball or the like placed on top of the post for such image-based reference markers. In this manner, the augmented reality display device 210 may be configured to determine the location from the known colours of the ping-pong balls and the orientation from the actual pixel locations of the ping-pong balls.

Now, in one embodiment, the system 200 comprises an interface computing device 205 in operable communication between the sensor 105 and the augmented reality display device 210 (or other integers of the system 200 such as the data server 220 or the like).

The interface computing device 205 may provide several advantages, including being able to communicate with the sensors 105 in accordance with a wireless communication protocol of network 180a being different to the wireless communication protocol network 180b. For example, the interface computing device 205 may communicate with the sensors 105 using the ZigBee protocol and communicate with the augmented reality display device 210 using the 802.11 protocol.

It should be noted that the interface computing device 205 may be left in place or alternatively portable and carried about by the vigneron such as by being a small form battery power computing device which the vigneron may carry in his pocket.

The interface computing device 205 further provides memory storage advantages wherein the interface computing device 205 comprises sufficient memory so as to be able to record a larger amount of data as would be possible for storage by each sensor 105.

In one embodiment, the interface computing device 205 comprises an inbuilt web server such that a standardised computing device display device 210 may simply browse to a particular IP address hosted by the interface computing device 205 to view the soil moisture content measurements. In this manner, practically any display computing device 210 having web browser functionality may interface using Wi-Fi or the like with the interface computing device 205.

The system 200 further comprises a centralized data server 220 adapted for the storage and propagation of soil moisture content data. Such data server 220 may be a remote web server operably coupled to the interface computing device 205, the augmented reality display device 210, each individual sensor 105 or the web browser client computing device 225.

In this manner, a number of soil moisture content measurements may be stored “in the cloud” for later retrieval by the relevant vigneron or even shared amongst authorised users.

The soil moisture content measurements may be made available by the data server 220 where the data server 220 has web server capabilities allowing for the viewing of the soil moisture content data using the web browser 225.

Furthermore, the data server 220 may act as an interface between the integers of the system 200 such as acting as an interface between the sensors 105 (or even the interface computing device 205) and the augmented reality display device 210. For example, the soil moisture content data as measured by the sensors 205 may be sent to the data server 220 for storage. Thereafter, the augmented reality display device 210 to retrieve the soil moisture content data upon request from the data server 220 for display.

As alluded to above, while modifications may be made to the technical architecture described herein within the purposive scope of the embodiments described herein, there will now be described an exemplary embodiment of a specific application of an embodiment of the system 200. It should be noted that this embodiment is exemplary only and no specific technical limitations should necessarily therefore be imputed thereto.

In this embodiment, the augmented reality display device 210 is in operable communication with the interface computing device 205. As alluded to above, the display device 210 is preferably adapted for augmented reality display of soil moisture measurement data however, the display of such data need not necessarily be augmented and may be displayed in other manners such as 2D representation, table representation and the like.

In the preferred embodiments, the interface computing device 205 is provided as a simple hardware accessory usable in conjunction with computing devices 210 compatible with many different types of operating systems e.g. Apple Inc. or Android devices, wherein the interface computing device 205 turns the computing device 210 into a gateway that send on the wireless-communication-enabled sensor's data to server 220 if a suitable data communications network is available.

In this example, the interface computing device 205 communicates with the sensors 105 using the Zigbee protocol. Furthermore, the interface computing device 205 communicates with the display device 210 using the Android Open Accessory Protocol (when accompanied with a computing device utilizing the Android operating system—it is further envisaged that the computing device may also utilize operating systems (e.g. iOS) associated with products offered by Apple Inc. or other available operating systems—e.g. Symbian and the like—through appropriate connection protocols as would be appreciated by the skilled addressee).

The following description is provided with the assumption that the sensor 105 is equipped with communications interface componentry compatible with the Zigbee communications protocol, however, it will be readily appreciated by the skilled addressee that sensor's using alternative network communications protocols are also applicable to the following discussion mutatis mutandis. The interface computing device 205 parses the Zigbee datastream emanating from the sensors 105 including obtaining information including the unique identifier uniquely identifying each sensor 105, the measurement of value and the sensor type.

The interface computing device 205 may take the form of an embedded computing device and may operate firmware written in C++.

The interface computing device 205 may be checked against previously received data in the memory device 110 of the interface computing device 205 and updated if necessary (i.e., on change). In this manner, the interface computing device 205 receives data from all of the sensors 105 along with a header.

For example, the header may comprise: streams start| reserved| number of packets| size of each packet| number of measurements, and therefore each of two received data packets for each sensor 105, the unique identifier| sensor type| value.

The display computing device 210 may be adapted to request measurement data from the interface computing device 205 at periodic intervals. In this manner, at a scheduled wake-up, the software application being executed by the display computing device 210 sends a message to the interface computing device 205 indicating that the display computing device 210 is ready for a set of measurements wherein the first computing device 205 then transmits the above-mentioned header and other information to the display computing device 210 using the Android Open Accessory Protocol. Once this has occurred, the memory device 110 of the interface computing device 205 is raised in anticipation of population with further sensor data, e.g. soil moisture content measurement data, obtained from incoming Zigbee packets.

Preferably, the display computing device 210 is a standard computing device, such as a mobile computing device or the like. In this regard, the display computing device 210 maybe operably coupled to the interface computing device by USB so as to allow the software application of the display computing device 210 to schedule regular wakeups such as by using the wakeful library. As such, upon wake-up, the display computing device request data from the interface computing device 205 using the Android Open Accessory Protocol. The interface computing device 205 responds with the above-mentioned header and other information which are parsed by the software application of the display device 210 and stored in a SQLite database.

The processed sensor information is then sent to the data server 210 in a standardized format such as, for example JSON format, should a data connection be available, such as a wireless or cellular data network. Once this has been accomplished, the software application of the display computing device 210 then reverts the display computing device 210 to standby. The advantage of utilizing a standardized data format allows interoperability of the system with existing sensor and interface technology which is adapted for communication via the selected data format. For instance any sensor (or gateway) that is able to send a JSON message to a RESTful API available on server 220 is compatible with the visualisation application on the computing device 210 as disclosed herein.

Using the display computing device 210 the vigneron is presented with a list of each sensor 105 and is able to view the soil moisture content data measured by each sensor 105. Preferably, the vigneron is able to view the soil moisture content data over time such as by using a suitable charting library or the like. As alluded to above, the vigneron may view the data using the display computing device 210 or a standard Web browser client computing device 225.

In particular arrangements of the telemetry system disclosed herein, it was found during initial developments stages that loss of communications connections was occurring between the interface computing devices 205. It was realized that this communication loss was due to operational characteristics using common and standard programming practices for the software code executed on the computing devices within the telemetry system. To overcome this loss of data communications, it was surprisingly realised that by creating a service that wakes the computing device 210 periodically and keeps device 210 locked in an awakened state until the following process is finished before being allowed to resume sleeping, device 210 polls the interface computing device 205 for the latest sensor data, stores the data on the computing device 210 and sends it on to server 220 if a communications network is available.

Preferably, the data stored on the data server 220 is correlated with the geographic location of the sensors 105. Such may be recorded using the display device computing device 210 during the initial installation or alternatively manually input in relation to each sensor 105. In this manner, soil moisture content data visualisations may be achieved in accordance with selected geographic regions.

It should be noted that variations may be made to the embodiments described herein within the purposive scope described herein. For example, embodiments that require real-time data or are data intensive wherein the sensor doesn't require much computation power would be in the use of the JavaScript (node.js) with internet protocol (IPv4 or IPv6) over wireless (802.11) on both the sensor 105 and the display computing device 210. This would obviate the need for an interface device where wireless coverage exists. Additionally it is possible to apply a MQ Telemetry Transport (MQTT) protocol interface on both the device and sensor 105 which would allow asynchronous communication with intensive computations run in the background. Additionally the serial version of the protocol, MQTT-S, can be used to do this with Zigbee communication.

The following definitions are provided as general definitions applicable to the interpretation of this specification and the accompanying claims and should in no way limit the scope of the present invention to those terms alone, but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (i.e. to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others. Thus, “including” is synonymous with and means “comprising”.

As used herein, the term “exemplary” is used in the sense of providing examples, as opposed to indicating quality. That is, an “exemplary embodiment” is an embodiment provided as an example, as opposed to necessarily being an embodiment of exemplary quality for example serving as a desirable model or representing the best of its kind.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the summary above and the description below, all transitional phrases such as “comprising,” “including,” “carrying,” “having,” “containing,” “involving,” “holding,” “composed of,” and the like are to be understood to be open-ended, i.e., to mean “including but not limited to”. Only the transitional phrases “consisting of” and “consisting essentially of” alone shall be closed or semi-closed transitional phrases, respectively.

For the purpose of this specification, where method steps are described in sequence, the sequence does not necessarily mean that the steps are to be carried out in chronological order in that sequence, unless there is no other logical manner of interpreting the sequence.

In addition, where features or aspects of the invention are described in terms of Markush groups, those skilled in the art will recognise that the invention is also thereby described in terms of any individual member or subgroup of members of the Markush group.

In the context of this document, the term “bus” and its derivatives, while being described in a preferred embodiment as being a communication bus subsystem for interconnecting various devices including by way of parallel connectivity such as Industry Standard Architecture (ISA), conventional Peripheral Component Interconnect (PCI) and the like or serial connectivity such as PCI Express (PCIe), Serial Advanced Technology Attachment (Serial ATA) and the like, should be construed broadly herein as any system for communicating data.

As described herein, ‘in accordance with’ may also mean as a function of and is not necessarily limited to the integers specified in relation thereto.

As described herein, ‘a computer implemented method’ should not necessarily be inferred as being performed by a single computing device such that the steps of the method may be performed by more than one cooperating computing devices.

Similarly objects as used herein such as ‘web server’, ‘server’, ‘client computing device’, ‘computer readable medium’ and the like should not necessarily be construed as being a single object, and may be implemented as a two or more objects in cooperation, such as, for example, a web server being construed as two or more web servers in a server farm cooperating to achieve a desired goal or a computer readable medium being distributed in a composite manner, such as program code being provided on a compact disk activatable by a license key downloadable from a computer network.

In the context of this document, the term “database” and its derivatives may be used to describe a single database, a set of databases, a system of databases or the like. The system of databases may comprise a set of databases wherein the set of databases may be stored on a single implementation or span across multiple implementations. The term “database” is also not limited to refer to a certain database format rather may refer to any database format. For example, database formats may include MySQL, MySQLi, XML or the like.

The invention may be embodied using devices conforming to other network standards and for other applications, including, for example other WLAN standards and other wireless standards. Applications that can be accommodated include IEEE 802.11 wireless LANs and links, and wireless Ethernet.

In the context of this document, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of this document, the term “wired” and its derivatives may be used to describe circuits, devices, systems, methods, techniques, communications channels, etc., that may communicate data through the use of modulated electromagnetic radiation through a solid medium. The term does not imply that the associated devices are coupled by electrically conductive wires.

Unless specifically stated otherwise, as apparent from the following discussions, it is appreciated that throughout the specification discussions utilizing terms such as “processing”, “computing”, “calculating”, “determining”, “analysing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer” or a “computing device” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. The processing system further may include a memory subsystem including main RAM and/or a static RAM, and/or ROM.

Furthermore, a computer-readable carrier medium may form, or be included in a computer program product. A computer program product can be stored on a computer usable carrier medium, the computer program product comprising a computer readable program means for causing a processor to perform a method as described herein.

In alternative embodiments or arrangements, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

Thus, one embodiment or arrangement of each of the systems and/or methods described herein is in the form of a computer-readable carrier medium carrying a set of instructions, e.g., a computer program that are for execution on one or more processors. Thus, as will be appreciated by those skilled in the art, embodiments or arrangements of the present invention may be embodied as a method, an apparatus such as a special purpose apparatus, an apparatus such as a data processing system, or a computer-readable carrier medium. The computer-readable carrier medium carries computer readable code including a set of instructions that when executed on one or more processors cause a processor or processors to implement a method. Accordingly, aspects of the present invention may take the form of a method, an entirely hardware embodiment, an entirely software embodiment or an embodiment or arrangement combining software and hardware aspects. Furthermore, the present invention may take the form of carrier medium (e.g., a computer program product on a computer-readable storage medium) carrying computer-readable program code embodied in the medium.

The software may further be transmitted or received over a network via a network interface device. While the carrier medium is shown in an example embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media.

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

Furthermore, some of the embodiments are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

Similarly, it is to be noticed that the term connected, when used in the claims, should not be interpreted as being imitative to direct connections only. Thus, the scope of the expression a device A connected to a device B should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other.

Reference throughout this specification to “one embodiment”, “one arrangement”, “an embodiment” or “an arrangement” means that a particular feature, structure or characteristic described in connection with the embodiment is included in at least one embodiment of the present invention. Thus, appearances of the phrases “in one embodiment/arrangement” or “in an embodiment/arrangement” in various places throughout this specification are not necessarily all referring to the same embodiment or arrangement, but may. Furthermore, the particular features, structures or characteristics may be combined in any suitable manner, as would be apparent to one of ordinary skill in the art from this disclosure, in one or more embodiments or arrangements.

Similarly it should be appreciated that in the above description of example embodiments or arrangement of the invention, various features of the invention are sometimes grouped together in a single embodiment/arrangement, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment or arrangement. Thus, the claims following the Detailed Description of Embodiments are hereby expressly incorporated into this Detailed Description of Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

It is apparent from the above, that the arrangements described are applicable to the telemetry systems industries.

It will be appreciated that the methods/apparatus/devices/systems described/illustrated above at least substantially provide an improvement over prior technology especially with regard to cost of product manufacture and customer experience.

The systems and methods described herein, and/or shown in the drawings, are presented by way of example only and are not limiting as to the scope of the invention. Unless otherwise specifically stated, individual aspects and components of the systems and methods described herein may be modified, or may have been substituted therefore known equivalents, or as yet unknown substitutes such as may be developed in the future or such as may be found to be acceptable substitutes in the future. The systems and methods described herein may also be modified for a variety of applications while remaining within the scope and spirit of the claimed invention, since the range of potential applications is great, and since it is intended that the present systems and methods described herein be adaptable to many such variations.

1. A telemetry system for distributed soil moisture content measurement, the system comprising:
a data network adapted for transmitting data;
a sensor operably coupled to the data network;
a computing device operably coupled to the data network adapted for providing an augmented reality display; and
an interface computing device adapted to communicate with the computing device,
wherein, in use:
the sensor is adapted to generate sensor data representing a measurement;
the interface computing device is adapted to receive, from the sensor, the measurement data and communicate the measurement data to the computing device; and
the computing device is adapted to process the measurement data received from the interface computing device; convert the measurement data into augmented reality data and to display the augmented reality data on a display module of the computing device representing an augmented reality display in accordance with the sensor data.

2. A telemetry system as claimed in claim 1, wherein the sensor is adapted for sensing moisture content.

3. A telemetry system as claimed in claim 1, wherein the augmented reality display represents the measurement.

4. A telemetry system as claimed in claim 3, wherein the augmented reality display represents a binary threshold representation of the measurement.

5. A telemetry system as claimed in claim 3, wherein augmented reality display represents a scalar representation of the measurement.

6. A telemetry system as claimed in claim 1, wherein, in use, the augmented reality display device is adapted to display the sensor data in accordance with a location of the sensor.

7. A telemetry system as claimed in claim 6, wherein, in use, the augmented reality display device is further adapted for receiving, from the sensor, location data representing a location of the sensor.

8. A telemetry system as claimed in claim 7, wherein, in use, the augmented reality display device is configured with a location of the sensor.

9. A telemetry system as claimed in claim 3, wherein, the augmented reality display device is adapted to display an interpolation of the measurement.

10. A telemetry system as claimed in claim 1, wherein the interface computing device is operably coupled to the data network.

11. A telemetry system as claimed in claim 10, wherein the interface computing device comprises memory for storing digital data and wherein, in use, the interface computing device is adapted to cache the sensor data.

12. A telemetry system as claimed in claim 10, wherein, in use, the interface computing device is adapted to establish communication with the augmented reality display computing device when the augmented reality display computing device is within proximity of the interface computing device and, having establish communication, transmit, to the augmented reality display computing device, sensor data from the memory.

13. A telemetry system as claimed in claim 10, wherein the interface computing device is adapted to interface with the sensor using a first wireless protocol and interface with the augmented reality display computing device using a second wireless protocol.

14. A telemetry system as claimed in claim 13, wherein the first wireless protocol is the ZigBee wireless protocol.

15. A telemetry system as claimed in claim 13, wherein the second wireless protocol is the 802.11 wireless protocol.

16. A telemetry system as claimed in claim 10, wherein the interface computing device comprises a web server and wherein, in use:
the interface computing device is adapted to receive an HTTP request from the augmented reality display computing device; and
the interface computing device is adapted to send, to the augmented reality display computing device, an HTTP response comprising the sensor data.

17. A telemetry system as claimed in claim 10, wherein, in use, the interface computing device is adapted to send, to a remote data server, the sensor data.

18. A telemetry system as claimed in claim 17, wherein, in use, the augmented reality display computing device is adapted to receive, from the data server, the sensor data.
